(19) United States
(12) Patent Application Publication
(10) Pub. No.: US 2022/0354755 A1
(43) Pub. Date: Nov. 10, 2022

(54) SPRAYABLE COSMETIC COMPOSITION COMPRISING POLYGLYCEROL ESTERS

(71) Applicant: Henkel AG & Co. KGaA, Duesseldorf (DE)

(72) Inventors: Thomas DOERING, Dormagen (DE); Daniel SOLICH, Langenfeld (DE)

(73) Assignee: Henkel AG & Co. KGaA, Duesseldorf (DE)

(21) Appl. No.: 17/735,667

(22) Filed: May 3, 2022

(30) Foreign Application Priority Data
May 4, 2021 (DE) 102021111500.0

(51) Int. Cl. A61K 8/04; A61K 8/85; A61Q 15/00

(52) U.S. Cl. CPC A61K 8/046; A61K 8/85; A61Q 15/00; A61K 2800/87

(57) ABSTRACT

The present disclosure relates to a sprayable composition and methods for using the sprayable composition. In one example, the sprayable composition includes ethanol, polyglycerol esters, water, and a blowing agent.

### CROSS-REFERENCE TO RELATED APPLICATION

This application claims priority to German Patent Application No. 102021111500.0, filed May 4, 2021, which is incorporated herein by reference in its entirety.

### TECHNICAL FIELD

The present disclosure relates to a sprayable cosmetic composition including polyglycerol esters, a cosmetic agent including said composition and the use of the composition as a deodorant.

The use of personal care products, especially fragrances, to combat unpleasant body odor has a long tradition. While perfume oils were used in ancient Egypt and antiquity, today deodorants are used, applied in the armpits to counteract unwanted perspiration odor. Deodorants are available in a variety of different dosage forms, such as spray, pump spray, roller, stick, soaked wipes, cream, powder or crystals.

### BACKGROUND

Administration in the form of a spray is usually conducted with the aid of propellants, i.e., gases that are compressed or pressure-liquefied to convey or atomize liquids or solid powders by employing their pressure. In this context, the hydrocarbons known collectively as CFCs, among others, have gained sad notoriety.

In the face of increasing environmental awareness, there is a desire to find alternatives to conventional blowing agents and to use inert gases such as nitrogen or compressed air as blowing agents. However, these systems show poorer aerosol formation compared to systems using LPG. Thus, the droplets present in the spray jet are already quite large at the beginning and continue to grow as the pressure drops. This results in an undesirable wet skin feeling for the user. In addition, larger droplets can fall out of the spray, an effect known as the “rainfall effect,” and thus do not reach their destination in the first place.

It is true that attempts were made to compensate for the disadvantages of inert gases as blowing agents by increasing the pressure. However, this measure has not proven to be practical in that the pressure inevitably drops with each use and, furthermore, an increase in pressure requires more robust aerosol containers, which in turn require more energy to manufacture and thus run counter to the goal of sustainability.

It is therefore desirable to provide a sprayable composition that overcomes the disadvantages of the prior art.

### BRIEF SUMMARY

The present disclosure relates to a sprayable composition and methods for using the sprayable composition. In an exemplary embodiment, the sprayable composition includes ethanol, polyglycerol esters, water, and a blowing agent.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It was surprisingly found that the addition of polyglycerol esters can reduce the droplet size in the spray stream. It was also possible to counteract an increase in droplet size with decreasing pressure and to achieve a pleasant skin feel across all applications.

Therefore, an embodiment of the present disclosure provides a sprayable cosmetic composition including:

a) ethanol;

b) polyglycerol esters;

c) water; and

d) a blowing agent.

Polyglycerol esters with a certain HLB value have proven to be particularly advantageous in the context of the present disclosure. The HLB value (HLB: hydrophilic-lipophilic balance) describes the hydrophilic and lipophilic fraction of molecules and can be determined, for example, using Griffin's method as follows:

$$H L B = 20 \times ( 1 - \frac{{M}_{l}}{M} )$$

where M₁ is the molar mass of the lipophilic portion of the molecule and M is the molar mass of the entire molecule and the factor 20 is a fixed scaling factor.

In the context of the present disclosure, polyglycerol esters having an HBL value of less than about 15 are particularly preferred. Therefore, an embodiment of the composition as contemplated herein is preferred in which the polyglycerol ester has an HBL value of about 9 to about 15, preferably about 11 to about 14, determined according to Griffin molecular weight calculation.

The present disclosure aims to provide a sprayable composition that has the same spray ability and droplet size properties as conventional compositions, while being more environmentally friendly. In a preferred embodiment of the composition as contemplated herein, the composition therefore uses as a blowing agent a compressed gas preferably selected from the group of air, nitric oxide, nitrogen, carbon dioxide and mixtures thereof. In a particularly preferred embodiment, nitrogen is used as the blowing agent.

The proportion of polyglycerol ester in the composition as contemplated herein is preferably about 0.05 to about 5 wt. %, particularly preferably about 0.2 to about 0.6 wt. %, in each case based on the total weight of components a) to c) of the composition as contemplated herein. Here it has been shown that such a proportion is sufficient to achieve the desired regulation of droplet size and thus a pleasant skin feel for the user.

Furthermore, it was surprisingly found that also a certain amount of water in the composition has a positive influence on the size of the droplets formed when spraying the composition as contemplated herein. Therefore, an embodiment is preferred in which the content of water in the composition as contemplated herein is about 0.5 to about 15 wt. %, preferably about 1 to about 5 wt. %, in each case based on the total weight of components a) to c) of the composition as contemplated herein.

The best results in terms of droplet size and spray ability of the composition as contemplated herein were obtained when it was alcohol-based. Therefore, the content of ethanol in the composition as contemplated herein, in each case based on the total weight of components a) to c) of the composition, is preferably at least about 70 wt. %, particularly preferably at least about 80 wt. %.

The desired effect concerning droplet size was observed more strongly in cases where the polyglycerol ester used had a chain length of the ester of up to 12 carbon atoms. Therefore, an embodiment in which the polyglycerol ester of the composition as contemplated herein includes an ester having a chain length of 8 to 12 carbon atoms is preferred. In a particularly preferred embodiment, the polyglycerol ester is polyglyceryl-3-caprylate. The composition as contemplated herein is particularly designed to be used to combat unpleasant body odors. Therefore, an embodiment in which the composition further includes fragrance is preferred.

The sprayable cosmetic composition of the present disclosure can be prepared in a conventional manner. All ingredients of the cosmetic composition except the propellant are placed in a suitable pressure-resistant container. This is then closed with a valve. Using conventional techniques, the desired amount of blowing agent is then added.

Aerosol dispensers are particularly suitable for dispensing and applying the sprayable composition. Thus, another embodiment of the present disclosure is a cosmetic agent including a composition according to the present disclosure and an aerosol delivery container. The term aerosol dispenser refers to pressure vessels that have a higher pressure inside than outside the vessel and from which a gas stream can be withdrawn via a valve. Aerosol dispensing containers, in other words, are pressurized containers by employing which a product, for example a cosmetic composition, can be dispensed through a valve by the internal gas pressure of the container.

Aerosol dispensers can be pressure-resistant containers made of metal (such as aluminum, tinplate or tin), protected or non-splintering plastic, or glass coated on the outside with plastic. Pressure and breakage resistance, corrosion resistance, ease of filling, as well as aesthetic considerations, handiness, printability, etc., all play a role in their selection. Special internal protective coatings ensure corrosion resistance to the composition made up in the pressure vessel. Particularly preferably, the valves used have an internally painted valve divider, with the paint and valve material being compatible with each other. If aluminum valves are used, their valve dividers can be coated on the inside with Microflex paint, for example. If tinplate valves are used as contemplated herein, their valve dividers can be coated with PET (polyethylene terephthalate), for example.

In accordance with the present disclosure, multi-chamber dispensers can also be used as aerosol dispensing containers. The multi-chamber dispenser may be configured such that one chamber includes the compressed propellant and another chamber is filled with the remaining ingredients of the composition as contemplated herein. One such multi-chamber dispenser is a so-called bag-in-can packaging.

Another embodiment of the present disclosure is the use of the sprayable cosmetic composition as contemplated herein as a deodorant.

The following examples are intended to further explain the subject matter of the present disclosure without limiting it in any way.

The following formulations were prepared and filled into aerosol delivery containers up to 50 to 70% of the rim volume and gassed with nitrogen or compressed air up to a pressure of 7 to 12 bar. The figures are in each case in wt. %.

TABLE 1

| | E1 | E2 | E3 | E4 | E5 | E6 |
|---|---|---|---|---|---|---|
| Ethanol (96%) | 91.81 | 92.05 | 93.0 | 91.81 | 91.81 | 91.81 |
| Triethyl citrate | 6.0 | 6.0 | 6.0 | 6.0 | 6.0 | 6.0 |
| Phenoxyethanol | 0.75 | 0.75 | 0.75 | 0.75 | 0.75 | 0.75 |
| Polyglyceryl-3- | 0.44 | 1.2 | 0.25 | 0.22 | — | — |
| caprylate | | | | | | |
| Polyglyceryl-4- | — | — | — | — | 0.44 | — |
| laurate | | | | | | |
| Polyglyceryl-2- | — | — | — | 0.22 | — | 0.44 |
| caprate | | | | | | |

Formulations E1 and E7 to Ell, summarized in Table 2, were filled into aerosol delivery containers (60% of the edge volume) and gassed with nitrogen up to a pressure of 9 bar. The containers were sprayed until only 10% of the original contents remained. Then, particle size measurement in the spray jet was performed by laser diffraction (Spraytech, Malvern International Ltd.). The D(v)50 value indicates that 50% of the sample volume is present in particles smaller than the specified value.

The data show that the use of polyglyceryl-3-caprylate leads to a significant reduction in the size of the droplets in the spray jet.

The data in the table refer to wt. % in each case.

TABLE 2

| | E1 | E7 | E8 | E9 | V |
|---|---|---|---|---|---|
| Ethanol (96%) | 91.81 | 91.81 | 91.81 | 91.81 | 92.25 |
| Triethyl citrate | 6.0 | 6.0 | 6.0 | 6.0 | 6.0 |
| Phenoxyethanol | 0.75 | 0.75 | 0.75 | 0.75 | 0.75 |
| Polyglyceryl-3-caprylate | 0.44 | — | — | — | — |
| (HLB 13) | | | | | |
| Polyglyceryl-3-oleate | — | 0.44 | — | — | — |
| (HLB 9.5) | | | | | |
| Polyglyceryl 10 palmitate | — | — | 0.44 | — | — |
| (HLB 15) | | | | | |
| Polyglyceryl 10 palmitate | — | — | — | 0.44 | — |
| (HLB 15) | | | | | |
| Perfume | 1.0 | 1.0 | 1.0 | 1.0 | 1.0 |
| Droplet size | | | | | |
| D(v)10 [μm] | 31 | 38 | 37 | 37 | 41 |
| D(v)50 [μm] | 62 | 69 | 69 | 68 | 75 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A sprayable cosmetic composition comprising:
a) ethanol;
b) polyglycerol esters;
c) water; and
d) a blowing agent.

2. The sprayable composition according to claim 1, wherein the polyglycerol ester has an HLB value of about 9 to about 15 determined according to molecular weight calculation.

3. The sprayable composition according to claim 1, wherein the blowing agent is a gas selected from the group of air, nitrogen oxide, nitrogen, carbon dioxide and mixtures thereof.

4. The sprayable composition according to claim 1, wherein the content of polyglycerol esters, based on the total weight of components a) to c) of the composition, is about 0.05 to about 5 wt. %.

5. The sprayable composition according to claim 1, wherein the content of water, based on the total weight of components a) to c) of the composition, is about 0.5 to about 15 wt. %.

6. The sprayable composition according to claim 1, wherein the content of ethanol, based also on the total weight of components a) to c) of the composition, is at least about 70 wt. %.

7. The sprayable composition according to claim 1, wherein the polyglycerol ester is polyglyceryl-3-caprylate.

8. The sprayable composition according to claim 1, wherein the composition further comprises fragrances.

9. A cosmetic agent comprising the sprayable composition according to claim 1 and an aerosol delivery container that contains the sprayable composition.

10. A method comprising the step of using the sprayable composition according to claim 1 as a deodorant.
